(19) United States
(12) Patent Application Publication
(10) Pub. No.: US 2007/0298084 A1
(43) Pub. Date: Dec. 27, 2007

(54) Flat System for Using in the Oral Cavity

(76) Inventors: Stefan Bracht, Gllenlcke-Nordbahn (DE); Babett Gutsmuths, Boehlen (DE)

Correspondence Address: Striker Striker & Stenby, 103 East Neck Road, Huntington, NY, 11743, US

(21) Appl. No.: 10/588,222

(22) PCT Filed: Feb. 3, 2005
(86) PCT No.: PCT/EP05/01076

§ 371 Date: Aug. 2, 2006

Related U.S. Application Data
(60) Provisional application No. 60/541,396, filed on Feb. 3, 2004.

(30) Foreign Application Priority Data
Apr. 2, 2004 (DE) 1020040170304

(51) Int. Cl. A61K 9/68; A61P 43/00

(52) U.S. Cl. 424/440

(57) ABSTRACT

The invention relates to a flat system for using in the oral cavity, and to a method for the production thereof. Said system consists of at least one upper water-soluble covering layer and at least one lower water-soluble covering layer. At least one intermediate layer is provided between the upper and lower covering layers, said intermediate layer having a smaller surface area than the covering layers and being recessed along the edge of the flat system.

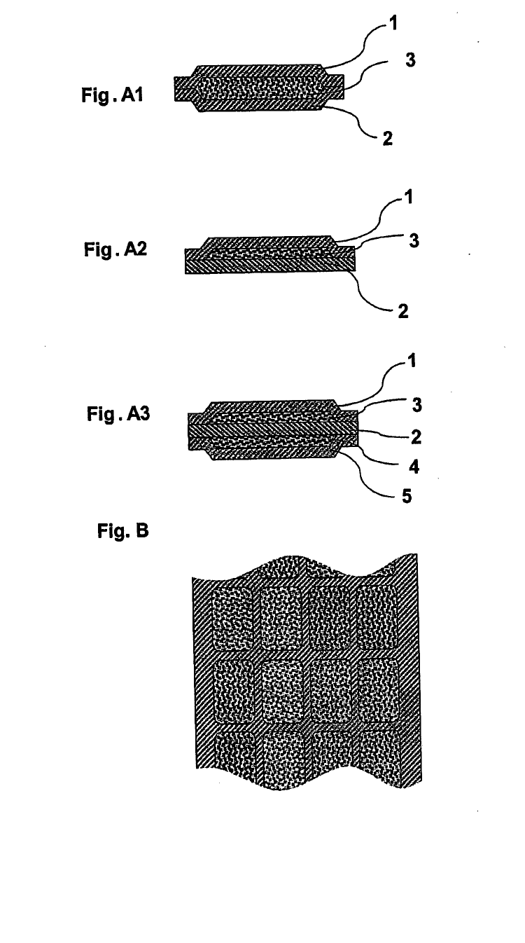

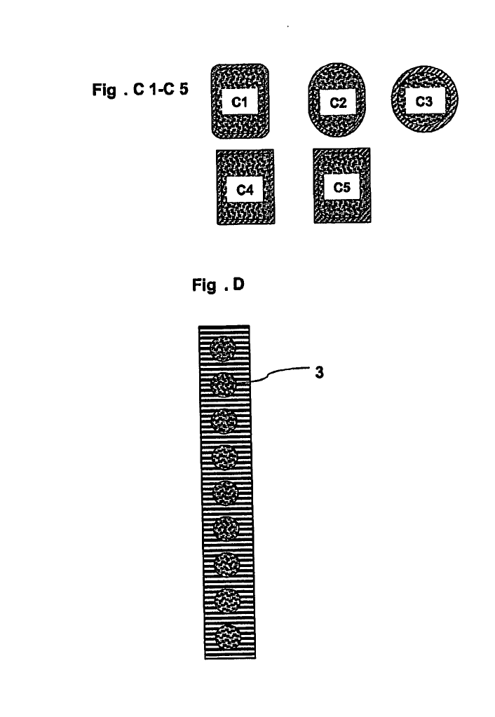

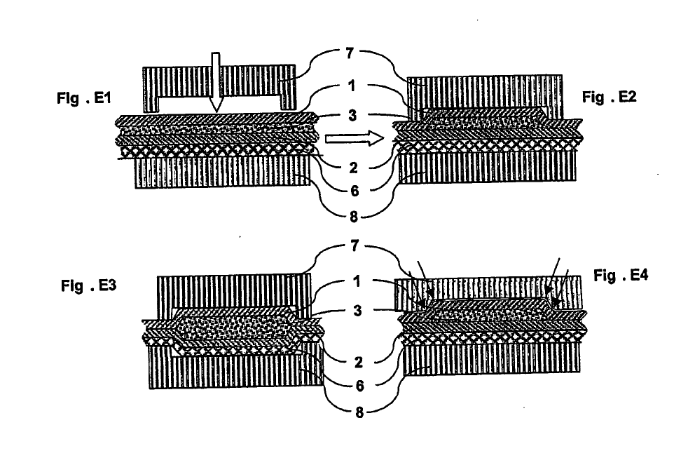

### TECHNICAL FIELD

The invention relates to a flat system for use in the oral cavity and a method for manufacturing it. This system is comprised of at least one upper and at least one lower water-soluble covering layer. Between the upper and lower covering layers, at least one intermediate layer is provided, which has a smaller area than the covering layers and is recessed along the edge of the flat system.

### PRIOR ART

Flat preparations for use in the oral cavity are known. Typically, these are water-soluble polymer films that break down rapidly in the mouth by dissolving in the saliva. The polymer film can contain ingredients intended for oral or dental care or for deodorizing, disinfecting, or freshening purposes, which essentially act in the oral cavity or in the nasal/throat cavity.

Products from this field include, for example, Eclipse Flash from the Wrigley Company or Listerine PocketPaks from the Pfizer Company.

In addition to cosmetic uses, pharmaceutical agents can also be contained in the flat preparations—such products are currently in development.

As examples of the prior art, reference is hereby made to the patents DE 2432925, DE 19956486A1, DE 19652257A1, DE 19652188, DE 10107659, and WO 03/011259 A1.

It is advantageous to be able to administer pharmaceutical products without drinking water, to eliminate the swallowing that some find unpleasant, and to offer numerous possibilities for the active ingredient to perform its function in the oral cavity or in the bloodstream via the oral cavity, for example through transmucosal absorption.

The disadvantages of flat drug formats according to the prior art are as follows:
water-soluble polymer films are manufactured from an aqueous solution, at temperatures typically above 100° C. and with relatively long drying times since the removal of water necessitated by its high thermal capacity in comparison to organic solvents is a process that consumes a lot of energy. These process conditions can be unsuitable for easily volatile or thermally unstable active ingredients,
or the active ingredients can easily decompose chemically at high temperatures in aqueous solution.
The flat drug formats typically contain the active ingredient distributed evenly over the surface. If shapes other than rectangular or square are used, then some of the active ingredient is wasted in the cutting process.
Loading with the active ingredient, as it increases in concentration, impairs the film-forming property of water-soluble polymers (e.g. increasing brittleness) and the loading capacity of the films is reduced by this effect.
The water-soluble films are manufactured using highly hydrophilic polymers that naturally have a low solubility for lipophilic active ingredients frequently encountered in pharmaceutical applications, e.g. steroid hormones. As a result, the films have a low loading capacity for lipophilic active ingredients, which can then only be loaded in the form of a crystal suspension or as multiphase systems, e.g. an emulsion.
The water-soluble polymer films are as a rule comprised of highly functional polymers that have a large number of hydroxyl or carboxyl functions in the polymer chain. These highly functional polymers are in a position to undergo numerous chemical interactions with pharmaceutical agents, which can easily lead to stability problems.
In order to be processable, water-soluble polymer films typically require a residual moisture content that assures sufficient flexibility or prevents brittleness. The residual water content, however, has a negative effect on the chemical stability of active ingredients in medicines.

### DESCRIPTION OF THE INVENTION

The object of the present invention, therefore, is to overcome these disadvantages of conventional flat pharmaceutical products for use in the oral cavity.

According to the present invention, this object is attained by means of a flat system for use in the oral cavity, comprised of at least one upper and at least one lower water-soluble covering layer; at least one intermediate layer is provided between the upper and lower covering layers. This intermediate layer has a smaller area than the covering layers because the intermediate layer is recessed along the edge of the flat system.

According to the present invention, the upper and lower covering layer can be attached to each other in a sealed fashion along the edge of the flat system.

The width of the sealed seam can be 0.3-3 mm, preferably 0.5-2 mm, and particularly preferably 0.75 to 1.5 mm.

In the flat system according to the present invention, the overall thickness of this flat system at its thickest point can be 50 to 500 μm, preferably 100 to 300 μm, and particularly preferably 150 to 250 μm.

According to the present invention, the intermediate layer can be water-soluble and have a melting point between 30 and 120° C., preferably between 50 and 100° C., and particularly preferably between 60 and 90° C.

The intermediate layer can also be water-insoluble.

Advantageous embodiments of the flat system according to the present invention are comprised of the solid preparation of the intermediate layer, which melts at temperatures between 30 and 45° C., preferably between 32 and 40° C., and particularly preferably between 35 and 38° C.

The intermediate layer can be comprised of a matrix that is used in the manufacture of rectal suppositories, preferably made of one or more hard fats (Adeps solidus) as described in the monograph of the European Pharmacopeia.

The intermediate layer can also be an oleaginous solution, suspension, or emulsion.

According to the present invention, the intermediate layer can be segmented within the flat product by virtue of the fact that the upper and lower covering layer are attached to each other in a sealed fashion in this region.

The intermediate layer can also contain at least one pharmaceutical agent in a dissolved or undissolved form.

Furthermore, the solubility of the pharmaceutical agent in the intermediate layer can be at least n times 10, preferably n times 10-100, where n represents the solubility of the covering layers.

A method for manufacturing a flat system is claimed in which, in a first method step, an intermediate layer composed of a lipophilic pharmaceutical preparation is deposited in a thin layer onto a water-soluble polymer layer and then covered with a second water-soluble polymer layer, whereupon in a subsequent method step, the upper and lower polymer layers are attached to each other in segments by means of heat sealing; mechanical pressure at the sealing points displaces the intermediate layer situated between the upper and lower polymer layer and the sealed covering layers form fully enclosed compartments in the intermediate layer.

In the method according to the present invention, the residual moisture in the water-soluble polymer films can be set to a value that improves the sealing capacity, preferably a value of 1-10% and particularly preferably 2-5% (m/m) water content.

In addition, in the method according to the present invention, the residual moisture in the water-soluble polymer films can be reduced by means of a drying process after the manufacture of the flat capsules.

In the method according to the present invention, the sealing capacity of the water-soluble polymer films can also be assured by means of softening additives from the group of hydrophilic fluids, preferably from the group of polyvalent alcohols with 3 to 6 carbon atoms (C₃- C₆), particularly preferably glycerol, 1,2-propylene glycol, 1,3-propylene glycol, 1,3-butane diol, hexylene glycol, or dipropylene glycol.

The flat system according to the present invention can contain one or two steroid hormones for hormone replacement therapy or for hormonal contraception.

The steroid hormones can be levonorgestrel, gestoden, dienogest, desogestrel, 3-keto-desogestrel, norelgestromin, drospirenon, estradiol, ethinyl estradiol, estradiol valerate, testosterone, testosterone undecanoate, testosterone enanthate, 7-alpha-methyl-19-nortestosterone, or its fluorine-containing derivatives.

In the flat system according to the present invention, an active ingredient can be selected from the group of organic nitrates (used to treat angina pectoris), in particular glycerol trinitrate, or an active ingredient from the group of antiemetics, in particular the 5-HT₃ receptor antagonists and particularly preferably from the group of ondansetron, granistron, ramosetron, alosetron, or the pharmaceutically acceptable salts thereof.

The flat system can also contain a nicotine base or a pharmaceutically acceptable salt thereof.

With both organic nitrates and nicotine, it is necessary to deliver the active ingredient into the bloodstream as quickly as possible directly via the oral mucous membranes.

It is also possible for the flat system according to the present invention to contain active ingredients for the treatment of geriatric illnesses, in particular morbus Alzheimer, morbus Parkinson, dementia-inducing diseases as well as active ingredients for treatment of severe psychiatric illnesses such as schizophrenia or psychoses. These therapy fields are partly distinguished by a reduced capacity or willingness to swallow, thus making it advantageous to administer drugs via the oral cavity.

Surprisingly, the stated object can be attained by selecting a flat product with a multilayered construction in which the function of the water-soluble polymer film and the function of the active ingredient substrate are provided separately in different layers; the active ingredient-containing layer is embodied in the form of an intermediate layer with an area smaller than the total area of the system due to the fact that the intermediate layer is recessed along the edge of the flat system.

In addition, the water-soluble polymer films surprisingly have the capacity to be processed in a heat-sealable fashion. This surprisingly turns out to be the case, even when a lipophilic, oleaginous, or wax-like intermediate layer is contained between these layers before the heat sealing.

By means of the present invention, it is possible to embed an intermediate layer in an envelope of hydrophilic, water-soluble polymer films, in a form similar to that of an extremely flattened capsule.

The intermediate layer can be comprised of a fluid, semisolid, or wax-like solid preparation. When used in the oral cavity, first, the envelope of water-soluble polymer films dissolves. Then, the intermediate layer breaks down by melting, by dissolving in the saliva, or by the two processes occurring simultaneously.

In the case in which the intermediate layer melts, an embodiment is preferred in which the compound melts at typical oral cavity temperatures of between 32 and 37° C.

In this way, the intermediate layer is practically imperceptible to the user and the mouthfeel is significantly more pleasant than that of an intermediate layer that remains solid. This also facilitates and accelerates the melting-induced release of the active ingredient from the lipophilic layer. With the use of wax-like intermediate layers, melting should not yet occur at temperatures below 30° C. in order to prevent the melting from occurring while the drug is being stored.

Suitable materials for the manufacture of the external water-soluble covering layers include water-soluble polymers from the group of polyvinyl alcohols with a hydrolysis grade of 75-99% (e.g. Mowiol® types), polyvinyl pyrrolidone, hydrophilic cellulose derivatives such as hydroxypropyl cellulose, hydroxymethylpropyl cellulose, or carboxymethyl cellulose, pullulan or maltose, hydrophilic starch derivatives such as carboxymethyl starch, alginates or gelatines, and other polymers known from the prior art.

The formulation and process engineering treatment of the intermediate layer are essentially determined by three requirements:
1. The intermediate layer must quickly dissolve in the mouth by melting or dissolving in the saliva or a combination of the two.
2. In the preferred case, the intermediate layer is applied in a coat directly onto a water-soluble polymer layer and, from a process-engineering standpoint, should require no solvents that can dissolve the polymer layer functioning as the coating substrate.
3. The intermediate layer must be thermoplastically deformable in order to be able to recede during production of the heat seal between the covering layers.

A wax-like, low-melting formulation is a preferable possibility for the formulation of the lipophilic intermediate layer. An example of this is the manufacture of rectal suppositories or vaginal suppositories. A selection of low-melting matrixes with melting points that can be selected from within a wide range can be made from the group of Softisan® and Witepsol® hard fats. Suitable substrate substances are also described in the monograph “Hard Fat” (Adeps solidus) of the European Pharmacopeia.

Alternatively, oleaginous, viscous solutions can be used as the intermediate layer. Suitable substrate substances include pharmaceutically common oils and lipophilic fluids that are preferably largely flavor-neutral, e.g. saturated triglycerides (e.g. Miglyol 812), isopropyl myristate, or isopropyl palmitate. Thickening agents can be added to these oleaginous solutions to increase the viscosity. With no claim as to completeness, these preferably include polymers from the group of polyacrylates (e.g. Eudragit® E 100 or Plastoid ® B), polyvinyl pyrrolidone (Kollidon® 25, 30, 90, or VA 64), polyvinyl acetate (e.g. Kollidon® SR), polyethylene glycol, or lipophilic cellulose derivatives (e.g. cellulose ethyl ether or cellulose acetate butyrate).

With no claim as to completeness, suitable polymer components of the intermediate layer include, for example, polyvinyl pyrrolidone (PVP) or its copolymers, e.g. Kollidon® 25, 30, 90, or VA 64, and polyethylene glycols (macrogols), with molecule masses of greater than 2000 Da.

It is also possible to add to the formulation of the intermediate layer, with no claim as to completeness, additives from the groups of softeners, tensides, solutizers, penetration improving agents, parting compounds, antioxidants, light and UV-protection agents, pigments, colorants, flavor correctants, organic or inorganic fillers and fragrances.

In this connection, the solutizers and penetration-improving agents are of particular importance:

On the one hand, the flat capsules according to the present invention have only a small interior volume, which reduces their loading capacity for active ingredients. Furthermore, it can be advantageous if the active ingredients contained are already completely or predominantly absorbed via the mucous membranes in the mouth instead of being absorbed only after being swallowed into the gastrointestinal tract.

The formulation of the intermediate layer should have the highest possible dissolving power for the active ingredient provided and solutizers can be used to this end. The solutizers must be selected so that they do not threaten the integrity of the water-soluble covering layers as a result of solubilizing, dissolving, or powerful softening.

Suitable solutizers include, for example, fatty acid esters of saturated fatty acids with chain lengths of 6 to 18 carbon atoms with monovalent to trivalent aliphatic alcohols having 2 to 4 carbon atoms (e.g. ethyl oleate, propylene glycol monolaurate, glycerol monooleate), fatty alcohol ethers of fatty alcohols having 6 to 18 carbon atoms with polyethylene glycol (e.g. BRIJ® products), fatty acid esters of fatty acids having 6 to 18 carbon atoms with polyethylene glycol (e.g. MYRJ® products), esters of fatty alcohols having 6 to 18 carbon atoms with carboxylic acids having 2 to 3 carbon atoms (lauryl lactate or lauryl acetate), sorbitan fatty acid esters (e.g. SPAN® products), sorbitan polyethylene glycol ethers of fatty acid esters (e.g. TWEEN® products), citric acid esters (e.g. triethyl citrate or acetyl tributyl citrate), diethylene glycol monothethylether (Transcutol®), propylene carbonate, solketal, glycofurol, tracetin, and cyclodextrine.

The compounds of the intermediate layer and the covering layers are advantageously selected so that in the intermediate layer, the solubility of the active ingredient in the intermediate layer is significantly greater than in the covering layers. This reduces potential undesirable chemical decomposition reactions of the active ingredient after migration into the covering layers.

To manufacture the flat systems according to the present invention, first, a water-soluble polymer film is manufactured by applying a solution in a coat onto a web-like substrate material and subsequently drying it. Alternatively, the film can also be manufactured by means of a solvent-free hot melting process.

The weight per unit area of the polymer layer is 25-200 g/m², preferably 40 to 150 g/m², and particularly preferably 60 to 100 g/m².

An intermediate layer is applied onto this starting material (polymer film on a substrate material, e.g. nonstick coated paper), from the side of the water-soluble polymer. This intermediate layer is preferably comprised of a medium-viscosity lipophilic fluid or the molten mass of a lipophilic compound. The lipophilic fluid or compound can be applied, for example, with the aid of a sheet die, a coating knife, a roller application unit, or a knife caster.

The weight per unit area of this intermediate layer is 25-300 g/m², preferably 30 to 200 g/m² , and particularly preferably 40 to 150 g/m².

The intermediate layer is preferably not deposited all the way up to the edge of the underlying polymer layer; instead, a margin of at least 0.5 to 5 cm is left at the edge in order to prevent the intermediate layer from coming out at the edge in the subsequent process steps.

The exposed surface of the intermediate layer, once it has resolidified after cooling, is covered with a second water-soluble polymer layer that as a rule, is of the same composition and produced with the same manufacturing method as the lowermost, first polymer layer. Preferably, however, the second water-soluble polymer layer is first removed from its substrate material and then laminated as a single layer onto the intermediate layer.

In a second process step, the composite made up of the substrate material, first water-soluble polymer layer, lipophilic intermediate layer, and second water-soluble polymer layer is subjected, with a suitable sealing mask, to a heat sealing coming from the uppermost, freely exposed polymer layer and consequently from the side furthest from the web-shaped substrate material.

The intermediate layer between the water-soluble polymer films is first melted as needed and then displaced by mechanical pressure at the parts to be sealed until the two water-soluble polymer films are joined to each other at these points with a permanent bond by means of heat sealing.

For heat-sealing capacity purposes, it can be advantageous to maintain a residual moisture in the water-soluble polymer films or to first adjust it by moistening.

The sealing capacity of the water-soluble polymer films can also be increased by means of softening additives from the group of hydrophilic fluids, preferably by means of additives from the group of multivalent alcohols with 3 to 6 carbon atoms (C₃-C₆), particularly preferably glycerol, 1,2-propylene glycol, 1,3-propylene glycol, 1,3-butane diol, hexylene glycol, or dipropylene glycol.

In cases in which a residual moisture is required for the sealing capacity, it can be necessary to dry the product after manufacture and to set the residual moisture to a lower value than required during sealing in order, for example, to increase the long-term chemical stability of the product.

In order to manufacture individually dosed formats, the upper and lower polymer layers are sealed together along a provided contour line so that a quantity of the lipophilic intermediate layer defined by the area is completely enclosed in individual dose fashion.

The individual doses of the active ingredient-containing intermediate layer are formed in this process step, which means that the sealing mask used must have corresponding dimensional accuracy of plus or minus 5% or better in order to be able to maintain the pharmaceutically required dosing accuracy.

With regard to the design of the sealing masks, it is advantageous to round the edges of the sealing contour in order not to exert unnecessarily high shear forces on the typically rather brittle water-soluble polymer films.

Finally, the manufactured flat products are mechanically cut or punched out along the sealing seams and thus divided up into individual forms or groups of individual forms.

The remaining widths of the sealed edge regions of the flat products should be kept as narrow as possible since in these regions, the water-soluble polymer films of the upper and lower covering layer jointly compose a particularly thick zone that can be expected to have the slowest dissolving speed in the mouth and therefore to have a negative effect on the mouthfeel. The width of the sealed seam should be 0.3 to 3 mm, preferably 0.5 to 2 mm, and particularly preferably 0.75 to 1.5 mm.

### DESCRIPTION OF THE FIGURES

FIG. A1 shows a schematic, cross-sectional view of a flat system: An upper covering layer (1) and a lower covering layer (2) cooperate to enclose an internal intermediate layer (3). In the case of FIG. A1, the two outer layers have a flat cavity, whereas in FIG. A2, the cavity for accommodating the intermediate layer is provided only in one of the two covering layers. The covering layers (1) and (2) can be identical or have differing structures.

FIG. A3 shows a flat system equipped with two separate chambers (3 and 4) formed with the aid of an additional covering layer (5).

FIG. B shows a flat intermediate product after the heat-sealing step; both longitudinally and transversely, the flat product can have a number of separate, active ingredient-containing segments of the intermediate layer that are transformed into the end products in additional process steps by being cut or punched out. FIGS. C1 through C5 show top views of various embodiment forms of the flat product according to the present invention. They are essentially intended to illustrate the possible embodiment forms. While the products C2 and C3 are more visually acceptable to consumers, the products C1, C4, and C5 have a higher coefficient of utilization of the flat intermediate product and produce less waste; this waste, however, does not contain any active ingredient. By contrast with FIG. C4, FIG. C5 illustrates the possibility that the contour line of the inner, active ingredient-containing intermediate layer does not have to follow the outer contour line of the flat product.

FIG. D shows an example of a flat product with a number of active ingredient-containing segments (3). This product represents a multi-dose product that can be divided up into individual doses.

FIGS. E1 through E4 illustrate a heat-sealing process step according to the present invention. A stamping, heat-sealing tool (7) presses the laminate—which is comprised of an upper covering layer (1), a lower covering layer (2), and an internal intermediate layer (3) on a web of substrate material (6)—against a counterpressure surface (8) of the sealing station; either the sealing tool alone or both the sealing tool and the counterpressure plate are heated.

This process step can also be executed using a counterpressure plate equipped with a flat cavity, which produces an approximately symmetrical cross-sectional form of the flat system according to the present invention. FIG. E3 schematically depicts the result of such a sealing procedure.

In FIG. E4, rounded regions are provided at the locations on the sealing mask (7) indicated with arrows, which regions lead to a reduction of the mechanical deformation stress on the covering layer (1) and therefore to a reduction in the risk of tears or leaks in the sealing seam. This technique can naturally also be applied to the two-sided form according to FIG. E3.

FIGS. E1 through E4 illustrate the relevant process step on a flatbed sealing die for a discontinuous operation in which the laminate web is brought to a halt during the process step. Naturally, this process step can also be executed on rotary systems equipped with correspondingly contoured sealing and embossing rollers and a continuously traveling laminate web.

For the primary packaging of the systems according to the present invention, reference is hereby made to the patents DE 19800682, DE 10008165, DE 10144287, DE 10102818, DE 10159746A1, and DE 10143120A1 and to the prior art cited therein.

### Exemplary Embodiments

### EXAMPLE 1

### Three-Layer Flat Capsule with a Wax-Like Semisolid Intermediate Layer

Intermediate layer: semisolid

Materials: Eclipse™ peppermint strips (3×2 cm) (Wrigley)

Softisan 100 (hard fat) (Sasol)
temperature-controlled water bath
beaker
single-use Pasteur pipettes
sealing pliers
   Implementation: a lipophilic intermediate layer

Softisan 100 is heated in the water bath until a clear molten mass forms. With the aid of a Pasteur pipette, the Softisan 100 is applied uniformly to the entire Eclipse™ peppermint strip (3×2 cm). After the hard fat begins to solidify, another Eclipse™ peppermint strip (3×2 cm) is placed precisely onto the lipophilic layer. The three-layer intermediate product is then sealed for approx. 5 sec. on all four sides with the aid of sealing pliers heated to approx. 160° C.

### EXAMPLE 2

### Five-Layer Flat Capsule with 2 Semisolid Intermediate Layers

Softisan 100 is heated in the water bath until a clear molten mass forms. With the aid of a Pasteur pipette, Softisan 100 is applied uniformly to the entire Eclipse™ peppermint strip (3×2 cm). After the hard fat begins to solidify, another Eclipse™ peppermint strip (3×2 cm) is placed precisely onto the lipophilic layer. A second hard fat layer is applied, which, after it solidifies, is in turn covered precisely with an Eclipse™ peppermint strip (3×2 cm). The five-layer intermediate product is then sealed for approx. 8 sec. on all four sides with the aid of sealing pliers heated to approx. 160° C.

### EXAMPLE 3

### Three-Layer Flat Capsule with an Oleaginous Intermediate Layer

Materials: Eclipse™ peppermint strips (3×2 cm) (Wrigley Co.)

viscous paraffin
single-use Pasteur pipettes
sealing pliers
   Execution:

With the aid of a Pasteur pipette, viscous paraffin is applied uniformly to the entire Eclipse™ peppermint strip (3×2 cm). Another Eclipse™ peppermint strip (3×2 cm) is placed precisely onto the lipophilic layer. The three-layer intermediate product is then sealed for approx. 5 sec. on all four sides with the aid of sealing pliers heated to approx. 160° C.

Definitions and calculations used in examples 1-3:

In order to determine the weight per unit area (FG), the manufactured multilayered products are individually weighed and the respective areas are determined. The weight of 10 Eclipse™ peppermint strips is determined and the average is calculated. The dimensions are correspondingly determined and the area is calculated.

The conversion of the units is taken into account in the calculation formula.
Strips:

$${FG}_{b} = \frac{{m}_{b}}{A} ⋆ 10$$

FG_{b}: weight per unit area (g/m²)
m_{b}: mass (average) (mg)
A: area (cm²)
FlatCaps:

$${FG}_{fc} = \frac{{m}_{fc}}{A} ⋆ 10$$
FG_{fc}: weight per unit area (g/m²)
m_{fc}: mass (average) (mg)
   lipophilic intermediate layer: FG=FG_{fc}-FG_{b}

In the examples, values of between 45 and 55 g/m² were determined for the weight per unit area of the Eclipse™ peppermint strips.

The weight per unit area of the Softisan layer in example 1 was 132 g/m².

The weight per unit area of the oleaginous layer in example 3 was 80 g/m².

What is claimed is:

1. A flat system for use in the oral cavity, comprised of at least one upper and at least one lower water-soluble covering layer; between the upper and lower covering layers, at least one intermediate layer is provided, wherein the intermediate layer has a smaller area than the covering layers due to the fact that the intermediate layer is recessed along the edge of the flat system.

2. The flat system as recited in claim 1,
wherein the upper and lower covering layer are attached to each other by means of sealing in the edge region of the flat system.

3. The flat system as recited in claim 1,
wherein the width of the sealed seam is 0.3-3 mm, preferably 0.5-2 mm.

4. The flat system as recited in claim 1,
wherein the overall thickness of the flat system at its thickest point is 50 to 500 μm, preferably 100 to 300 μm.

5. The flat system as recited in claim 1,
wherein the intermediate layer is water-soluble and has a melting point between 30 and 120° C., preferably between 50 and 100° C.

6. The flat system as recited in claim 1,
wherein the intermediate layer is water-insoluble.

7. The flat system as recited in claim 6,
wherein the intermediate layer is a solid preparation, which melts at temperatures between 30 and 45° C., preferably between 32 and 40° C.

8. The flat system as recited in claim 7,
wherein the intermediate layer is comprised of a matrix that is used in the manufacture of rectal suppositories, preferably made of one or more hard fats (Adeps solidus) as described in the monograph of the European Pharmacopeia.

9. The flat system as recited in claim 6,
wherein the intermediate layer is an oleaginous solution, suspension, or emulsion.

10. The flat system as recited in claim 1,
wherein the intermediate layer is segmented within the flat product by virtue of the fact that the upper and lower covering layer are sealed to each other in this region.

11. The flat system as recited in claim 1,
wherein the intermediate layer contains at least one pharmaceutical agent in a dissolved or undissolved form.

12. The flat system as recited in claim 11,
wherein the solubility of the pharmaceutical agent in the intermediate layer is at least n times 10, preferably n times 10-100, where n represents the solubility of the covering layers.

13. A method for manufacturing a flat system as recited in claim 1,
wherein
an intermediate layer composed of a lipophilic pharmaceutical preparation is deposited in a thin layer onto a water-soluble polymer layer,
then is covered with a second water-soluble polymer layer,
the upper and lower polymer layers are attached to each other in segments by means of heat sealing;
mechanical pressure at the sealing points displaces the intermediate layer situated between the upper and lower polymer layer and the sealed covering layers form fully enclosed compartments in the intermediate layer.

14. The method as recited in claim 13,
wherein the residual moisture in the water-soluble polymer films is set to a value that improves the sealing capacity, preferably a value of 1-10% (m/m) water content.

15. The method as recited in claim 14,
wherein the residual moisture in the water-soluble polymer films is reduced by means of a drying process after the manufacture of the flat capsules.

16. The method as recited in claim 13,
wherein the sealing capacity of the water-soluble polymer films is assured by means of softening additives from the group of hydrophilic fluids, preferably from the group of polyvalent alcohols with 3 to 6 carbon atoms (C₃- C₆), particularly preferably glycerol, 1,2-propylene glycol, 1,3-propylene glycol, 1,3-butane diol, hexylene glycol, or dipropylene glycol.
